SEQUENCE LISTING (4 sequences)

SEQ ID NO: 1 - DNA, 23 bp, Artificial Sequence
  Primer sequence used for RT-qPCR, p62 forward.
gaagaatgtgggggagagtgtgg

SEQ ID NO: 2 - DNA, 22 bp, Artificial Sequence
  Primer sequence used for RT-qPCR, p62 reverse.
tgcctgtgctggaactttctgg

SEQ ID NO: 3 - DNA, 21 bp, Artificial Sequence
  Primer sequence used for RT-qPCR, B2m forward.
cagcatggctcgctcggtgac

SEQ ID NO: 4 - DNA, 21 bp, Artificial Sequence
  Primer sequence used for RT-qPCR, B2m reverse.
cgtagcagttcagtatgttcg

(19) United States
(12) Patent Application Publication
(10) Pub. No.: US 2021/0145833 A1
(43) Pub. Date: May 20, 2021

(54) Methods and Compositions for Hair Growth by Activating Autophagy

(71) Applicant: The Regents of the University of California, Oakland, CA (US)

(72) Inventors: Jing Huang, Los Angeles, CA (US); Min Chai, Los Angeles, CA (US); Karen Lynn Reue, Torrance, CA (US); Laurent Vergnes, Los Angeles, CA (US); Gay Miriam Crooks, Los Angeles, CA (US); Stephanie Cochonneau de Barros, Los Angeles, CA (US); Meisheng Jiang, Thousand Oaks, CA (US)

(21) Appl. No.: 17/047,522

(22) PCT Filed: Apr. 15, 2019
(86) PCT No.: PCT/US2019/027430

§ 371 Date: Oct. 14, 2020

Related U.S. Application Data
(60) Provisional application No. 62/658,113, filed on Apr. 16, 2018.

(51) Int. Cl. A61K 31/517; A61K 31/7056; A61K 31/155; A61K 31/436; A61P 17/14; A61K 9/00; A61K 9/06

(52) U.S. Cl. CPC A61K 31/517; A61K 31/7056; A61K 31/155; A61K 9/0053; A61P 17/14; A61K 9/0014; A61K 9/06; A61K 31/436

(57) ABSTRACT

Disclosed are methods improving or stimulating hair regeneration; treating, inhibiting, or reducing hair loss; improving or stimulating hair growth; treating, inhibiting, or reducing pigmentation loss; and/or improving or stimulating pigmentation production in a subject with one or more autophagy inducing agents.

### CROSS-REFERENCE TO RELATED APPLICATIONS

This application claims the benefit of U.S. Patent Application No. 62/658,113, filed Apr. 16, 2018, which is herein incorporated by reference in its entirety.

### ACKNOWLEDGEMENT OF GOVERNMENT SUPPORT

This invention was made with Government support under Grant Numbers HL090553 and AG049753, awarded by the National Institutes of Health. The Government has certain rights in the invention.

### REFERENCE TO A SEQUENCE LISTING SUBMITTED VIA EFS-WEB

The content of the ASCII text file of the sequence listing named “20190415_034044_183WO1_seq_ST25” which is 1.13 kb in size was created on Apr. 15, 2019, and electronically submitted via EFS-Web herewith the application is incorporated herein by reference in its entirety.

### BACKGROUND OF THE INVENTION

The biological and psychological importance of hair is well recognized. Mammalian hair growth consists of cyclic repetitions of telogen (quiescence), anagen (regeneration) and catagen (degeneration) phases of the hair follicle. This hair follicle cycle is regulated by both intrinsic and extrinsic signals which control quiescence and activation of hair follicle stem cells (HFSC). Inadequate HF SC activation and proliferation underlie alopecia in numerous biological and pathological conditions, including aging. Molecules that can promote HF SC activation and anagen initiation have been intensely searched for, as they may both help reveal how hair regeneration is regulated and provide therapeutic and cosmetic interventions.

As a fundamental process for degrading and recycling cellular components, autophagy is critical for adaptation to nutrient starvation and other adverse environmental conditions as well as it is regulated by such signals. Autophagy is also important for quality control of proteostasis through the elimination of misfolded or damaged proteins and damaged organelles. The loss of autophagy may be causally related to neurodegeneration and other diseases. Autophagy declines with age, likely contributing to the higher prevalence of autophagy related diseases (e.g., cancer and neurodegenerative diseases) in the elderly. Autophagic clearing of active, healthy mitochondria in hematopoietic stem cells is required to maintain quiescence and stemness, and autophagy fulfills the nutrient demand of quiescent muscle stem cell activation. In the skin, autophagy is required for self-renewal and differentiation of epidermal and dermal stem cells, but its role in hair follicle stem cells has remained controversial. On one hand, autophagy may be required for hair growth as skin grafts from the autophagy related gene 7 (Atg7) deficient mice exhibit abnormal hair growth. On the other hand, psychological stress may induce autophagy and delay of hair cycle.

Hair loss or alopecia affects millions worldwide and can occur because of aging, hormonal dysfunction, autoimmunity, or as a side effect of cancer treatment. Methods and compositions that can be used to regrow hair are highly sought after, but lacking.

### SUMMARY OF THE INVENTION

In some embodiments, the present invention is directed to a method of stimulating hair regeneration in a subject in need thereof, which comprises administering to the subject one or more autophagy inducing agents. In some embodiments, the present invention is directed to a method of stimulating new hair growth in a subject in need thereof, which comprises administering to the subject one or more autophagy inducing agents. In some embodiments, the present invention is directed to a method for treating, inhibiting, or reducing hair loss in a subject which comprises administering to the subject one or more autophagy inducing agents. In some embodiments, the present invention is directed to a method for improving or stimulating hair growth in a subject which comprises administering to the subject one or more autophagy inducing agents. In some embodiments, the present invention is directed to a method for treating, inhibiting, or reducing pigmentation loss in a subject which comprises administering to the subject one or more autophagy inducing agents. In some embodiments, the present invention is directed to a method for improving or stimulating pigmentation production in a subject which comprises administering to the subject one or more autophagy inducing agents. In some embodiments, the hair loss is a result of the subject aging. In some embodiments, the pigmentation loss is a result of the subject aging. In some embodiments, the subject is aging and/or the subject is an aged subject. In some embodiments, the one or more autophagy inducing agents are administered in a therapeutically effective amount. In some embodiments, the therapeutically effective amount is administered as several doses over a given period of time, e.g., a daily dose for a week or more.

In some embodiments, the one or more autophagy inducing agents are administered to an area on the subject where new hair growth is desired. In some embodiments, the area has an amount of hair that is less than the amount present at an earlier period. In some embodiments, the area is absent of hair. In some embodiments, the area is absent of hair due to a disease or condition that decreases or inhibits hair growth. In some embodiments, the area is absent of hair due to an injury. In some embodiments, the area is absent of hair due to chemotherapy and/or radiation therapy. In some embodiments, the area is absent of hair due to surgery. In some embodiments, the subject has a thyroid disorder. In some embodiments, the subject has a pituitary gland disorder. In some embodiments, the subject has alopecia areata. In some embodiments, the subject has anagen effluvium and/or telogen effluvium.

In some embodiments, the therapeutically effective amount is administered as a single dose. In some embodiments, the therapeutically effective amount is administered in at least two doses, at least three doses, at least four doses, at least five doses, or more. In some embodiments, the therapeutically effective amount is administered daily. In some embodiments, the therapeutically effective amount is administered every other day.

In some embodiments, the method further comprises administering to the subject a supplementary agent. In some embodiments, the supplementary agent comprises one or more growth factors. In some embodiments, the growth factor comprises TGF-β2, IGF-1, KGF, or HGF. In some embodiments, the supplementary agent is administered in combination with the one or more autophagy inducing agents. In some embodiments, the supplementary agent is administered sequentially with the one or more autophagy inducing agents. In some embodiments, the supplementary agent and the one or more autophagy inducing agents are administered as a unified dosage form. In some embodiments, the supplementary agent and the one or more autophagy inducing agents are administered as separate dosage forms. In some embodiments, the dosage form is formulated for stimulating a cell to enter into anagen phase.

In some embodiments, the number of hair follicles in the subject after administration of the one or more autophagy inducing agents is higher relative to the number of hair follicles in the subject prior to administration of the one or more autophagy inducing agents. In some embodiments, the weight of a hair in the subject after administration of the one or more autophagy inducing agents is greater relative to the weight of a hair in the subject prior to administration of the one or more autophagy inducing agents. In some embodiments, the hair shaft length of a hair in the subject is increased faster after administration of the one or more autophagy inducing agents relative to the hair shaft length of a hair in the subject prior to administration of the one or more autophagy inducing agents. In some embodiments, the growth rate of a hair in the subject is increased after administration of the one or more autophagy inducing agents relative to the growth rate of a hair in the subject prior to administration of the one or more autophagy inducing agents. In some embodiments, the subject is a human.

In some embodiments, the one or more autophagy inducing agents are administered in the form of a composition. In some embodiments, the one or more autophagy inducing agents are formulated for oral, parenteral, or topical administration. In some embodiments, the one or more autophagy inducing agents are formulated for topical administration. In some embodiments, the one or more autophagy inducing agents are formulated as a gel. In some embodiments, the one or more autophagy inducing agents are formulated as a cream. In some embodiments, the one or more autophagy inducing agents are formulated as an ointment. In some embodiments, the one or more autophagy inducing agents are formulated as a lotion.

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description explain the principles of the invention.

### DESCRIPTION OF THE DRAWINGS

This invention is further understood by reference to the drawings wherein:

FIG. 1. Hair regeneration is induced by topical treatment with α-KG. Panel (A) Structural formula of α-KG. Panel (B) α-KG induces hair regeneration. Male mice were shaved on Postnatal Day 44 (telogen) and topically treated with vehicle control (DMSO in about 250 μL PLO Base) or α-KG (dissolved in DMSO and then added to about 250 μL PLO Base at 32 mM final) every other day over 39 days. Melanin pigmentation in the skin of α-KG treated animals, indicative of anagen induction by the treatment, became visible as early as on Day 12; vehicle-treated mice did not show significant pigmentation for at least 39 days. Hair growth from the pigmented skin areas of α-KG treated mice was visible within about 5-7 days. Photographs shown were taken on Day 39 post-treatment, by which time mice treated with α-KG exhibited overall hair growth whereas control mice still had no hair generally except for random hair patches on some animals. Total number of animals: control (32), α-KG (34). Similar effects by α-KG were seen in female mice (shown in FIG. 6, Panel D). Panel (C) Quantification for appearance of melanin pigmentation (indicating onset of anagen) in mouse skin treated with α-KG vs. control. Pigmentation scoring is described in Methods. Number of animals shown in Panel (A): control (4), α-KG (4). Panel (D) Microphotographs of hematoxylin and eosin (H&E) stained skin tissue sections from mice treated with 32 mM α-KG, showing new hair follicles and enlarged bulbs, elongated hair shafts, and thickened dermal layers. Hematoxylin is a basic dye that stains nucleic acids purplish blue; eosin is an acidic dye that stains cytoplasm and extracellular matrix (e.g., collagen) pink. Immunohistochemistry for Ki-67, a marker for cell proliferation, further demonstrated the formation of new hair follicles. IL-6 and F4/80 are inflammatory cytokine and macrophage markers, respectively. Controls for IL-6 and F4/80 positive inflammatory skin are shown in FIG. 6, Panel E. Panel (E) Induction of autophagy associated markers, including LC3, p62, and phosphorylated Beclin 1, in telogen skin of mice treated with α-KG for 6 hours, 24 hours, and 5 days. Skin remained in telogen during the treatment period as confirmed by the lack of skin pigmentation. Each lane is from a different animal. Number of animals: 4 for each treatment.

FIG. 2. Hair regeneration is induced by topical treatment with oligomycin and rapamycin. Panel (A) Structure of oligomycin. Panel (B) Oligomycin (100 μM) induces hair regeneration. Male mice were shaved on Postnatal Day 44 and topically treated every other day. Photographs shown were taken on Day 23 post-treatment. Number of animals: control (23), oligomycin (23). Similar effects by oligomycin were seen in female mice (FIG. 6, Panel D). Panel (C) Quantification for appearance of melanin pigmentation in mouse skin treated with oligomycin vs. control. Number of animals: control (3), oligomycin (3). Panel (D) Microphotographs of H&E and Ki-67 stained skin tissue section from mice treated with 100 μnM oligomycin. Panel (E) Western blot analysis of autophagy related markers in telogen skin of mice treated 5 days with indicated compounds. Ctrl, control; Oligo, oligomycin; Rapa, rapamycin. Panel (F) Structure of rapamycin. Panel (G) Rapamycin (1.6 μM) induces hair regeneration. Male mice were shaved on Postnatal Day 43 and treated topically every other day. Photographs were taken on Day 37 post-treatment. Number of animals: control (18), rapamycin (17). Rapamycin at 100 nM gave similar results as 1.6 μM (FIG. 7). Similar effects by rapamycin were seen in female mice (FIG. 7, Panel B). Rapamycin at 16 μM, however, resulted in hair loss and open wounds (data not shown) this may be due to more severe inhibition of mTOR which was reported to be required for HFSC activation. Panel (H) Quantification for appearance of melanin pigmentation in mouse skin treated with rapamycin (1.6 μM) vs. control. Number of animals: control (3), rapamycin (3). Panel (I) Microphotographs of H&E and Ki-67 stained skin tissue section from mice shown in Panel (G).

FIG. 3. Hair regeneration is induced by AICAR, metformin, and α-KB. Panel (A) Structure of AICAR. Panel (B) AICAR (16 mM) induces hair regeneration. Male mice were shaved on Postnatal Day 44 and treated topically every other day. Photographs were taken on Day 41 post-treatment. Number of animals: control (12), AICAR (11). Similar effects in females (not shown). Panel (C) Quantification for skin pigmentation in mice from Panel (B). Number of animals: control (3), AICAR (3). Panel (D) H&E and Ki-67 stained skin tissue section from mice treated with 16 mM AICAR. Panel (E) Structure of metformin. Panel (F) Metformin (160 mM) induces hair regeneration. Male mice were shaved on Postnatal Day 43 and treated topically every other day with metformin or vehicle control (H₂O in this experiment). Photographs were taken on Day 48 post-treatment. Number of animals: control (13), metformin (12). Similar effects in females (not shown).

Panel (G) Quantification for skin pigmentation in mice from Panel (F). Number of animals: control (3), metformin (3). Panel (H) H&E and Ki-67 stained skin tissue section from mice shown in Panel (F). Panel (I) Structure of α-KB. Panel (J) Oral α-KB (8 mM in drinking water) treatment negates hair loss in aged female mice. Photo was taken at 131 weeks of age. Number of animals: control (5), α-KB (5). Panel (K) Topical α-KB (32 mM) induces hair regeneration in young male mice. Mice were shaved on Postnatal Day 44 and treated topically every other day. Photographs were taken on Day 39 post-treatment. Number of animals: control (18), α-KB (18). Similar effects in females (not shown). Panel (L) Quantification for skin pigmentation in mice from Panel (K). Number of animals: control (4), α-KB (4). Panel (M) H&E and Ki-67 stained skin tissue section from mice treated with 32 mM α-KB. Panel (N) Western blot analysis of autophagy related markers in telogen skin of mice treated 5 days with indicated compounds.

FIG. 4. SMER28 induces hair regeneration in an autophagy-dependent manner. Panel (A) Structure of SMER28. Panel (B) Western blot analysis of autophagy related markers in telogen skin of mice treated 5 days with 1 mM SMER28. Each lane is from a separate mouse. Panel (C) Male mice were shaved on Postnatal Day 45 and treated daily with 1 mM SMER28; photographs were taken on Day 23 post-treatment. Every other day treatment demonstrated similar results (not shown). Number of animals: control (6), SMER28 (6). Similar effects were observed in females (not shown). Panel (D) SMER28 (2 mM) induced hair regeneration is inhibited by co-treatment with autophinib (4 mM). Mice were shaved on Postnatal Day 51 and topically treated every other day. Photographs were taken on Day 20 post-treatment; histology of corresponding skin tissue section was shown. Number of animals: control (20), SMER28 (16), SMER28+autophinib (7), autophinib (7).

FIG. 5. Autophagy levels are indicative of hair follicle cycle stages, increased upon anagen induction. Mice were shaved on Postnatal Day 93 for males and on Postnatal Day 92 for females and monitored for hair cycle progression. Mice at each indicated stage were sacrificed for Western blot analysis of autophagy markers. T, telogen; A, anagen; C, catagen.

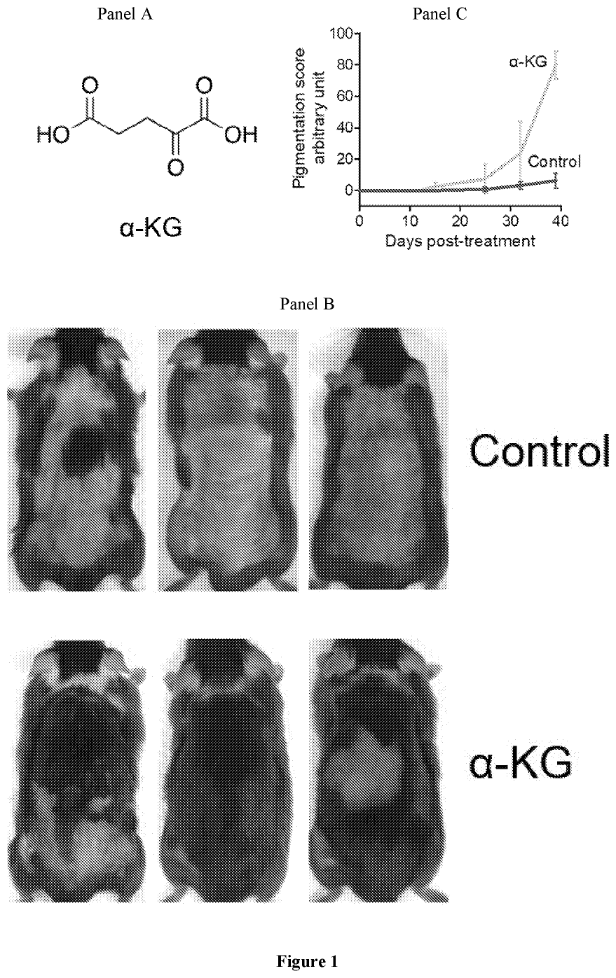

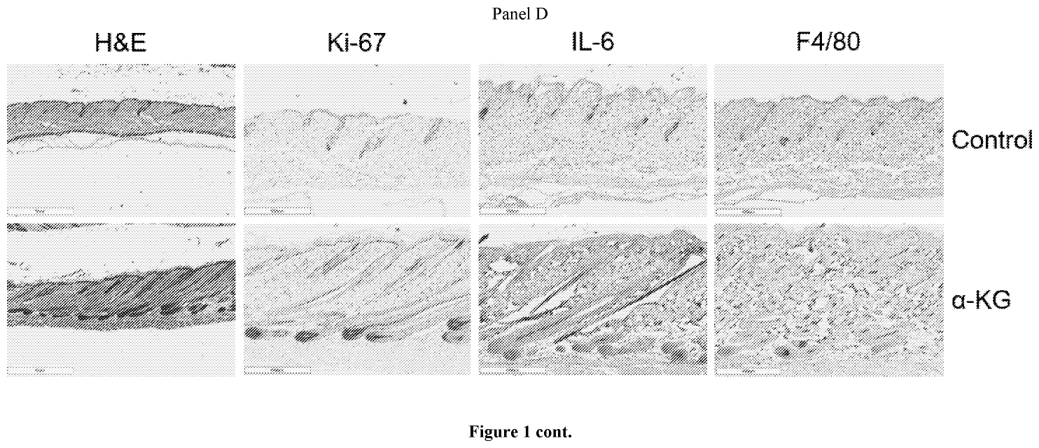

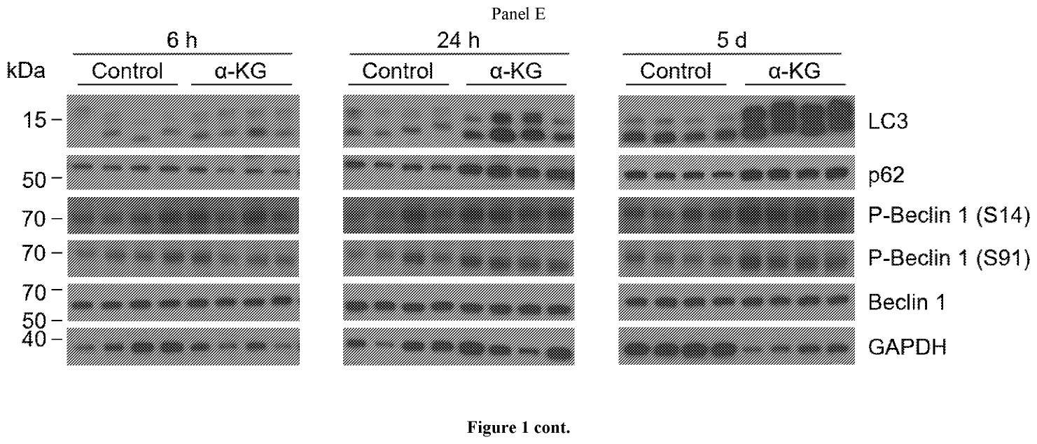

FIG. 6. In both male and female mice, hair regeneration can be induced by α-KG or oligomycin treatment. Related to FIG. 1. Panel (A) Minoxidil (5% in PLO base) as a positive control for hair regeneration. Photographs shown were taken on Day 22 post-treatment. Number of animals: control (3), minoxidil (3), α-KG (3). Panel (B) Compared to 6.5-week old mice in FIG. 1, Panel A, α-KG induces faster hair regeneration in 8-week old animals. Male mice were shaved on Postnatal Day 57 (telogen) and topically treated every other day with 32 mM α-KG. Photographs shown were taken on Day 20 post-treatment. Number of animals: control (4), α-KG (3). Panel (C) Quantification for skin pigmentation in mice from (A). Pigmentation of α-KG treated animals became visible as early as on Day 7, and full dorsal hair coverage was observed by Day 20 post-treatment. Number of animals: control (3), α-KG (3). Panel (D) α-KG and oligomycin also stimulate hair regeneration in female animals. Female mice were shaved on Postnatal Day 58 (telogen) and topically treated with vehicle control (DMSO), α-KG (16 mM), or oligomycin (10 μM) every other day. Photographs shown were taken on Day 26 post-treatment. Number of animals: control (9), α-KG (9), oligomycin (10). Panel (E) Positive controls showing IL-6 and F4/80 in damaged skin. 8-week old male mice with skin lesions, e.g., Fight wounds or bite lesions, were employed. Panel (F) Quantitative RT-PCR showing increased p62 transcript levels in α-KG (*P=0.026; by t-test, two-tailed, two-sample unequal variance) and SMER28 (*P=0.017; by t-test, two-tailed, two-sample unequal variance) treated skin. The housekeeping gene B2m (beta-2-microglobulin) was used as an internal control. Mean±standard deviation (s.d.) is plotted.

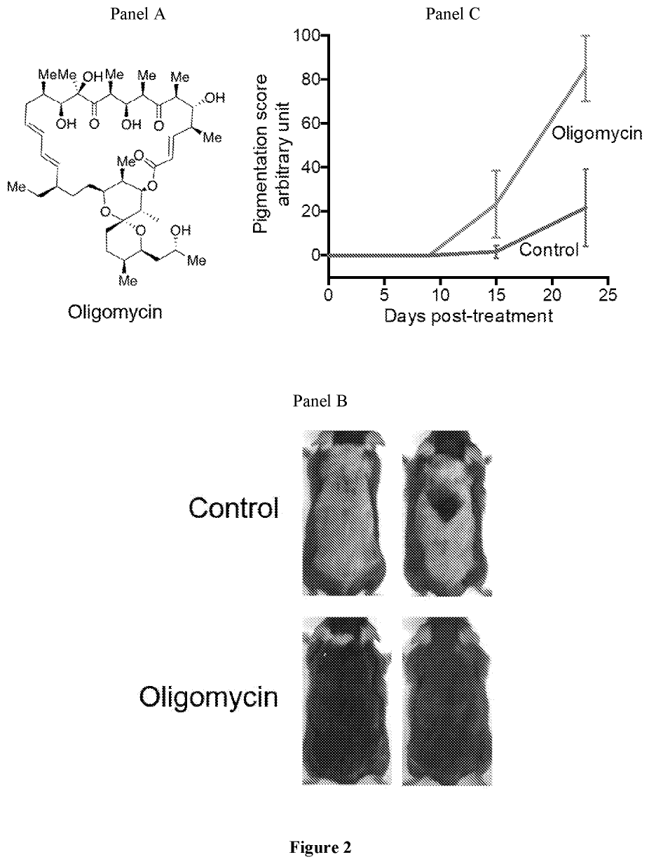

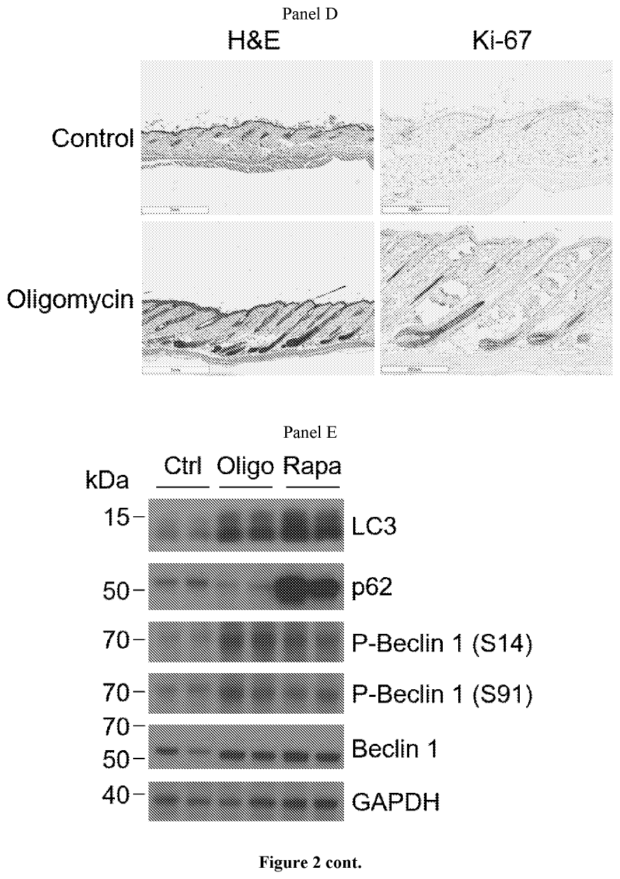

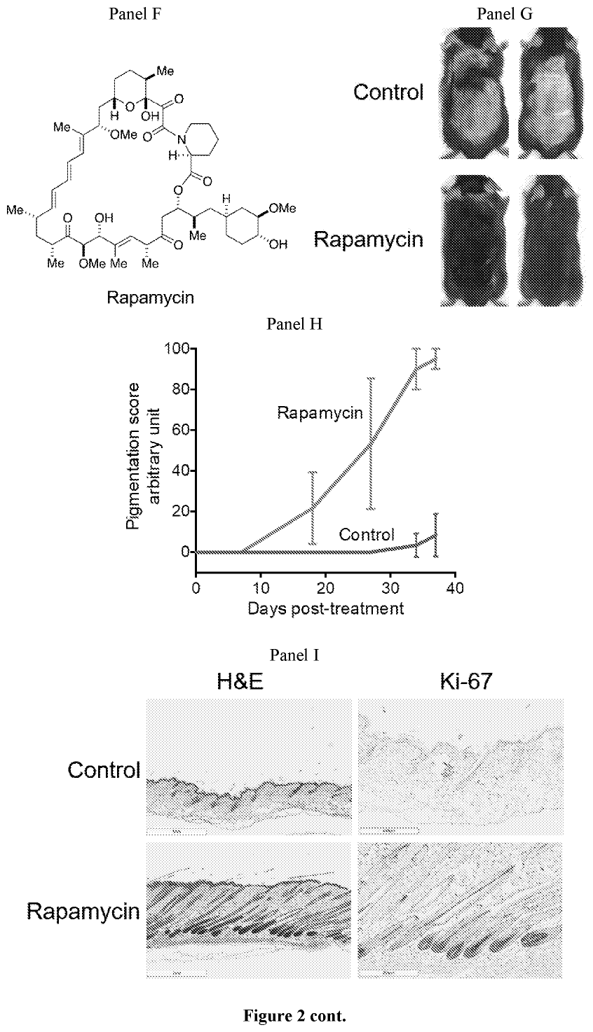

FIG. 7. Effects of rapamycin on hair regeneration. Related to FIG. 2. Panel (A) Rapamycin at 100 nM also promotes hair regeneration. Male mice were shaved on Postnatal Day 45 (telogen) and topically treated every other day with vehicle control (DMSO) or 100 nM rapamycin. Photographs shown were taken on Day 23 post-treatment. Number of animals: control (7), rapamycin (7). Panel (B) Rapamycin also promotes hair regeneration in female animals. Female mice were shaved on Postnatal Day 58 (telogen) and topically treated every other day with vehicle control (DMSO) or rapamycin (1.6 μM). Number of animals: control (9), rapamycin (11).

FIG. 8. Autophagy is required for α-KG to induce hair regeneration. Related to FIG. 4. Panel (A) Autophinib inhibits hair regeneration by α-KG. Male mice were shaved on Postnatal Day 53 (telogen) and topically treated with vehicle control (DMSO), autophinib (4 mM), α-KG (64 mM), or α-KG (64 mM) and autophinib (4 mM) together every other day. Photographs shown were taken on Day 20 post-treatment. Number of animals: 4 for each treatment. Panel (B) Western blot analysis of autophagy related markers in mouse skin on Day 5 post-treatment. Panel (C) Bafilomycin A1 also inhibits hair regeneration by α-KG. Male mice were shaved on Postnatal Day 52 (telogen) and topically treated with vehicle control (DMSO), bafilomycin (200 μM), α-KG (64 mM), or α-KG (64 mM) together with bafilomycin (200 μM) together every other day. Photographs shown were taken on Day 21 post-treatment. Number of animals: 4 for each treatment. FIG. 9. Images representing mouse hair cycle progression scores between 0 and 100. Related to FIG. 1 to FIG. 3. Mice were shaved and monitored for hair cycle progression. Arbitrary values from 0 to 100 were assigned based on skin pigmentation levels and hair shaft density, with 0 indicating no hair growth (and no pigmentation) and higher numbers corresponding to darker skin and larger areas of dense hair growth. For example, a score of 50 was assigned for full-length hair growth on 50% of dorsal skin area or pigmentation on 100% of dorsal skin area without hair shafts yet. A score of 70 was assigned for full-length hair growth on 70% of dorsal skin or pigmentation on 100% of dorsal skin with about 30-40% hair shafts. A value of 100 indicates full-length hair growth on 100% of dorsal skin.

### DETAILED DESCRIPTION OF THE INVENTION

Disclosed herein are compounds that induce autophagy and thereby stimulate hair regeneration (e.g., hair growth and hair follicle regeneration) in otherwise telogen skin. That is α-ketoglutarate (α-KG), α-ketobutyrate (α-KB), and drugs such as rapamycin and metformin which impinge on TOR and AMPK signaling induce autophagy and thereby stimulate hair regeneration. Stimulation of hair regeneration by these agents is blocked by specific autophagy inhibitors, suggesting a mechanistic link between autophagy and hair regeneration. Consistent with this idea, increased autophagy is detected upon anagen entry during the natural hair follicle cycle. The experiments herein indicate that forced induction of autophagy can activate resting telogen hair follicles.

As disclosed herein, rapid anagen entry on whole dorsal telogen skin was observed from time to time among mice treated with α-KG, oligomycin, rapamycin, and SMER28, but never in α-KB, metformin, or AICAR-treated mice, nor in vehicle control mice. Temporally, pigmentation (anagen entry) induction by α-KB, AICAR, or metformin takes much longer, e.g., on a time scale of about 12-18 days, as compared to about 5-14 days by α-KG, oligomycin, or rapamycin. It is possible that this may reflect a differential effect by mTOR inhibition and by AMPK activation on the regulation of autophagy. The crosstalk between metabolism and autophagy is complex. Autophagy is generally induced by limitations in ATP availability or a lack of essential nutrients, including glucose and amino acids, yet ATP is required for autophagy. Starvation and ensuing decreased energy charge and increased ROS levels are potent activators of autophagy.

Induction of autophagy is mediated by some of the same cellular energy metabolism regulators which have been linked to or implicated in the effect of dietary restriction (DR) on longevity. The metabolite α-ketoglutarate (α-KG) increases autophagy in both worms and cultured mammalian cells. Therefore, as disclosed herein, whether α-KG can stimulate hair regeneration was examined using an in vivo C57BL/6J mouse dorsal skin model. Minoxidil, a vasodilator used to treat pattern hair loss, was included for comparison as it has typically been used as a positive control in many papers on hair research (FIG. 6, Panel A). Male mice at 6.5 weeks of age (Postnatal Day 44) were shaved on the back, when dorsal skin hair follicles are in the telogen phase. α-KG or vehicle control treatment was applied topically every other day. α-KG treatment drastically enhances hair regeneration (FIG. 1, Panels A-B). Anagen in black mice is macroscopically recognizable by the melanin pigment visible through the skin, as the melanogenic activity of follicular melanocytes is strictly coupled to the anagen stage of the hair cycle. In the experiment shown in FIG. 1, Panel B, skin pigmentation was visible by Day 12 post-treatment with α-KG (FIG. 1, Panel C). In contrast, in vehicle-treated control mice no pigmentation or only a few scattered pigmented spots were apparent at least until Day 39, when animals were sacrificed for histological and biochemical analyses. Hair grew from the pigmented skin area of α-KG treated mice within about 5-7 days, and by Day 39 post-treatment α-KG treated mice exhibited robust hair growth; in contrast, control mice showed little or no hair growth overall (FIG. 1, Panel B). The effects of α-KG on anagen initiation and hair regeneration were even more dramatic when mice were treated later in telogen at 8 weeks of age (FIG. 6, Panels B-C). α-KG stimulation of hair growth is gender-independent; α-KG exhibited similar hair stimulating effects in female mice (FIG. 6, Panel D).

Formation and differentiation of hair follicles in α-KG treated mice were correspondingly demonstrated by histological analyses (FIG. 1, Panel D). More follicles and high proliferation marker Ki-67 expression were observed in α-KG treatment group, showing anagen phase induction (FIG. 1, Panel D). In telogen skin, α-KG initiated new anagen waves as early as Day 7 post-treatment. Since inflammation and wound repair are known to stimulate tissue, including hair regeneration, the experiments herein focused on molecules that do not cause skin damage or other abnormal skin conditions. There was no evidence of skin irritation or inflammation by α-KG or other small molecule treatments described in this study (unless otherwise indicated) as per visual inspection and confirmed by IL-6 and F4/80 staining (FIG. 1, Panel D and FIG. 6, Panel E).

Mice of the same age as those used for regeneration experiments were also acutely treated and analyzed for early biochemical changes. Increased autophagy induction in the α-KG treated mouse skin was supported by Western blot analysis of LC3, both at 24 hours and 5 days post-treatment (FIG. 1, Panel E). Expression of the autophagy substrate p62/SQSTM1, which is widely used as an indicator of autophagic degradation, was also increased with autophagy induction by α-KG in the mouse skin (FIG. 1, Panel E) as well as by rapamycin induced autophagy (see below). This is likely due to compensation through upregulation of p62 transcription (FIG. 6, Panel F).

Longevity increase by α-KG was found to be mediated, at the molecular level, through direct inhibition of the highly conserved mitochondrial ATP synthase/ATPase (complex V) and subsequent decrease of target of rapamycin (TOR) activity downstream. Whether hair regeneration by α-KG may also be mediated by ATP synthase inhibition was also examined. Topical treatment with the complex V inhibitor oligomycin similarly promoted hair regeneration in both male (FIG. 2, Panels A-D) and female (FIG. 6, Panel D) mice. Also, like α-KG, oligomycin treatment results in TOR inhibition and autophagy activation. Increased autophagy was detected in topical oligomycin treated mouse skin as indicated by LC3 expression (FIG. 2, Panel E).

The target of rapamycin (TOR) protein is a main mediator of the effect of DR in longevity. Inhibition of TOR, e.g., by rapamycin, elicits autophagy. Therefore, whether rapamycin increases hair regeneration was also examined. As shown in FIG. 2, Panel F-I and FIG. 7, topical rapamycin treatment accelerated hair regeneration as determined by both visual and histological methods. Autophagic LC3, p62, and mTOR-dependent phosphorylated Beclin 1 S14 were increased in rapamycin treated mouse telogen skin (FIG. 2, Panel E). Consistently, Beclin 1 S14 phosphorylation was also increased in mouse skin by Day 5 post-treatment with α-KG (FIG. 1, Panel E) and oligomycin (FIG. 2, Panel E). Together, these results show that hair regeneration can be accelerated by either indirect or direct inhibition of TOR pathway activity and induction of autophagy.

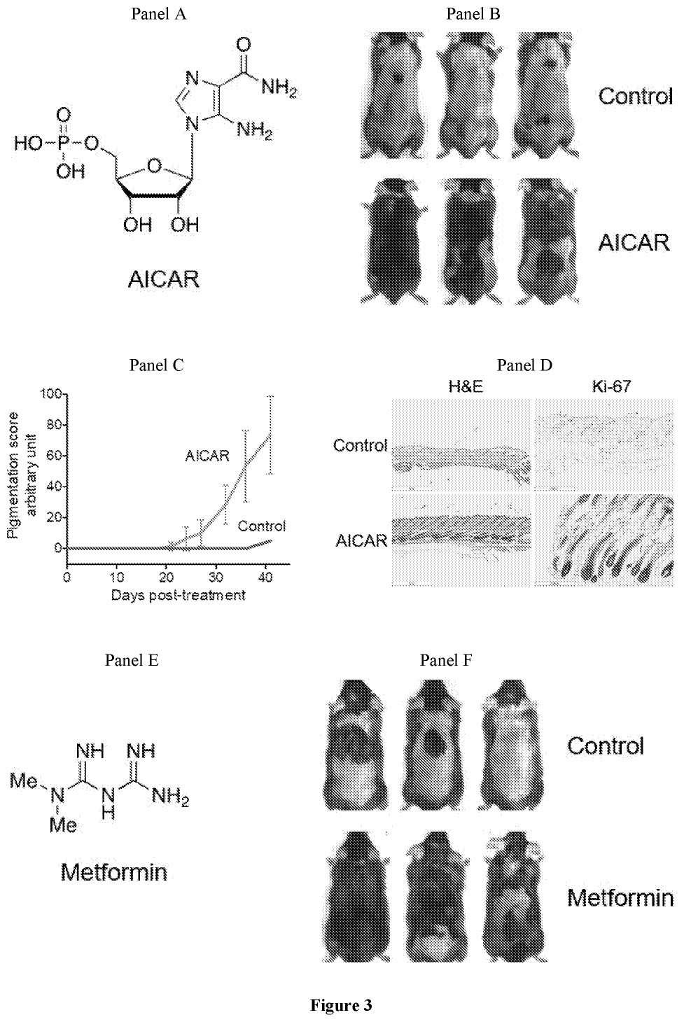

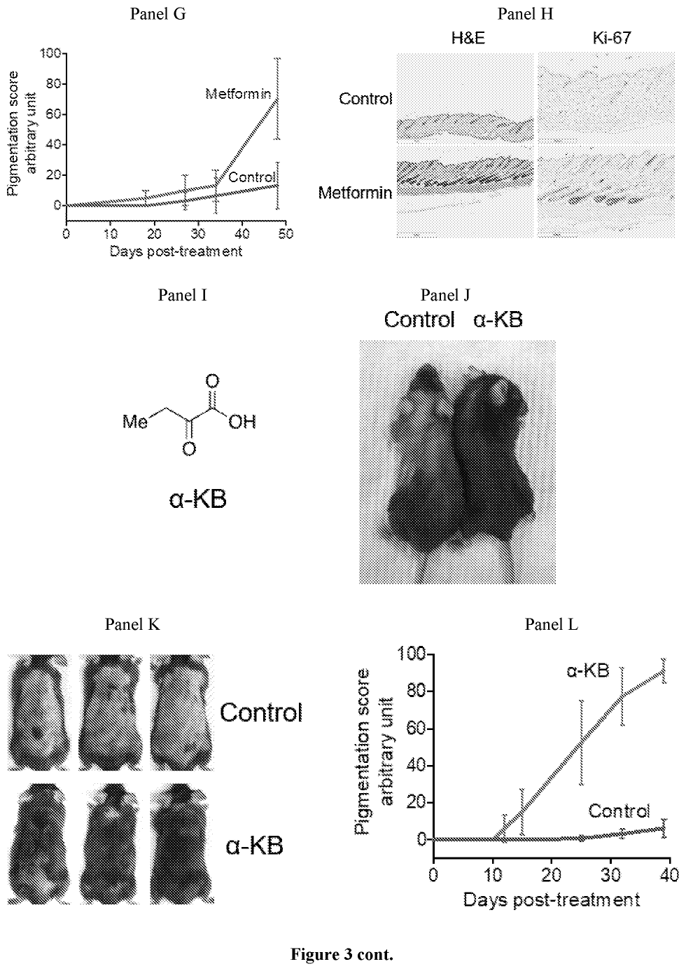

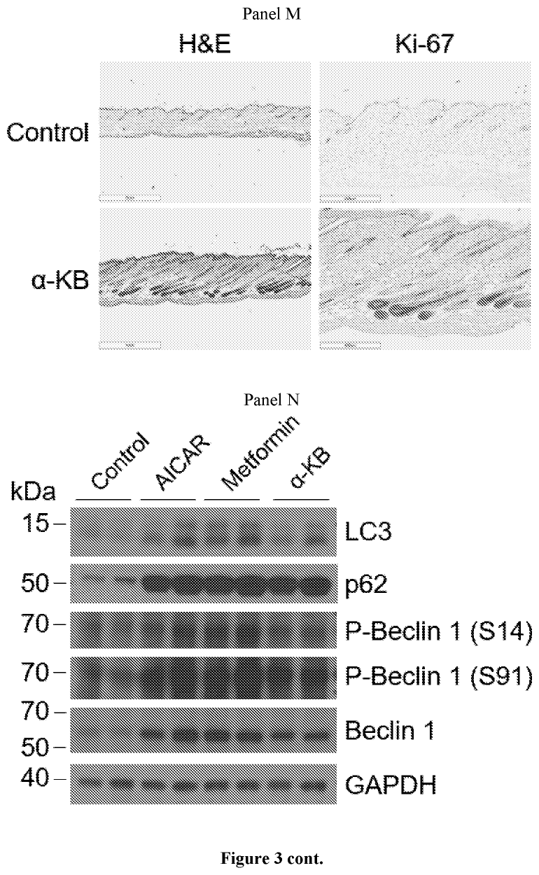

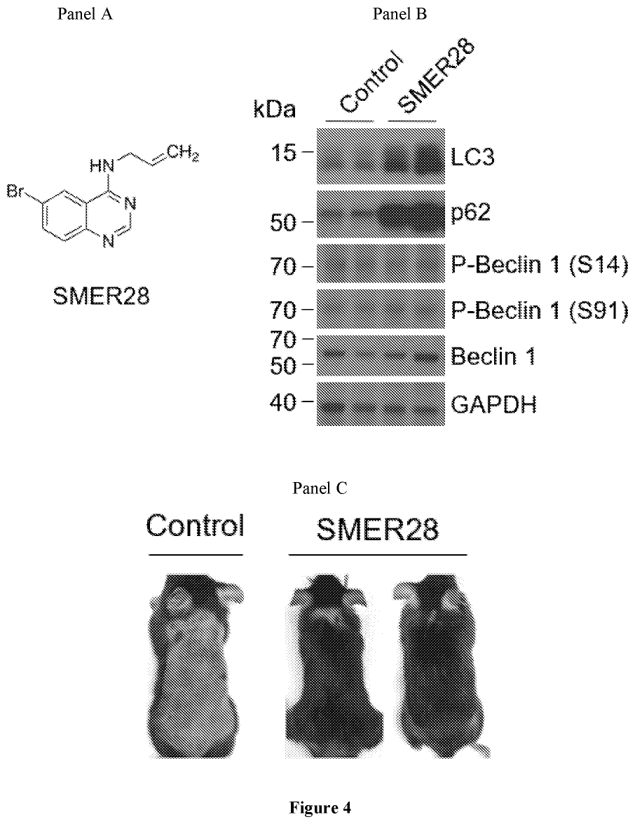

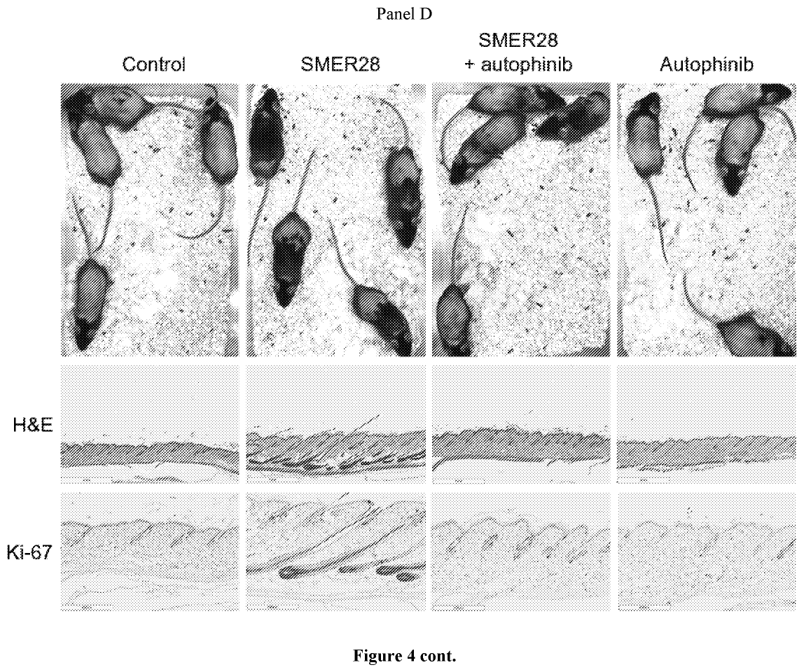

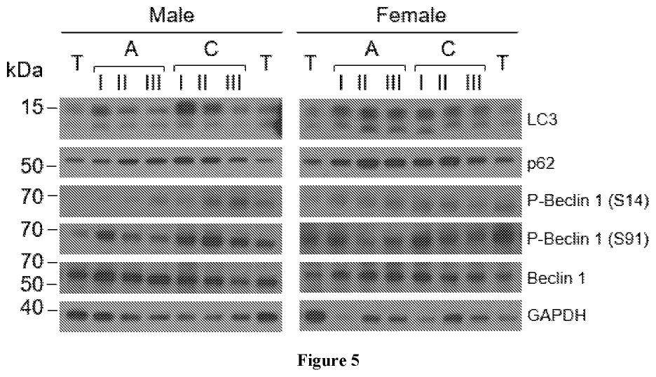

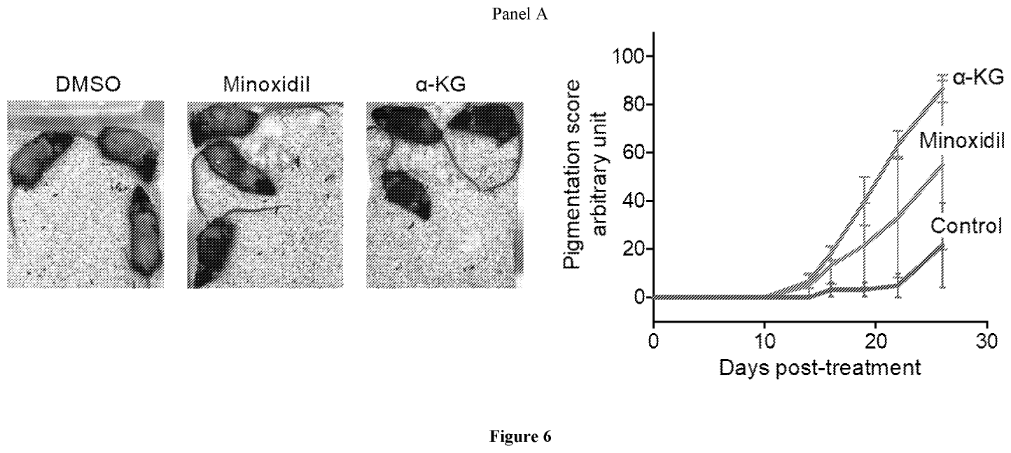

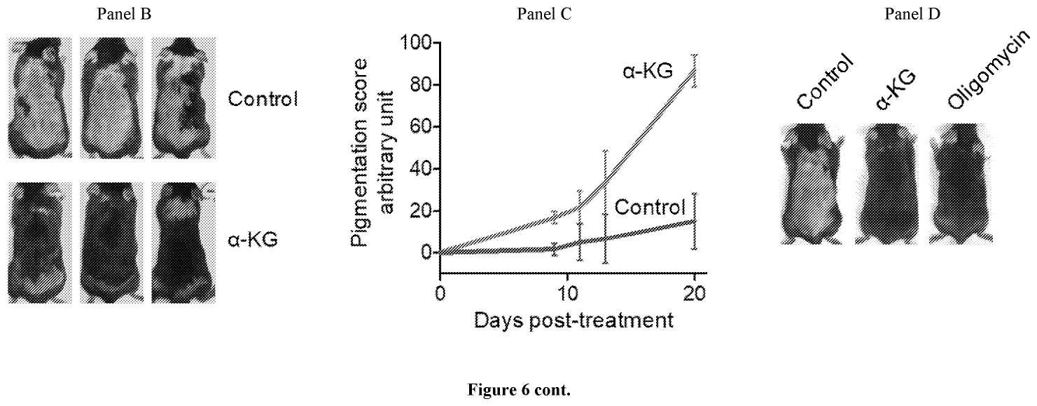

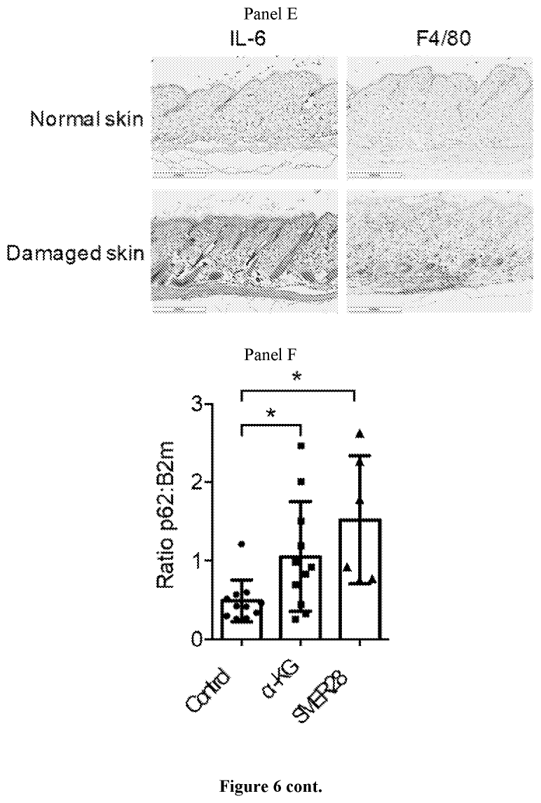

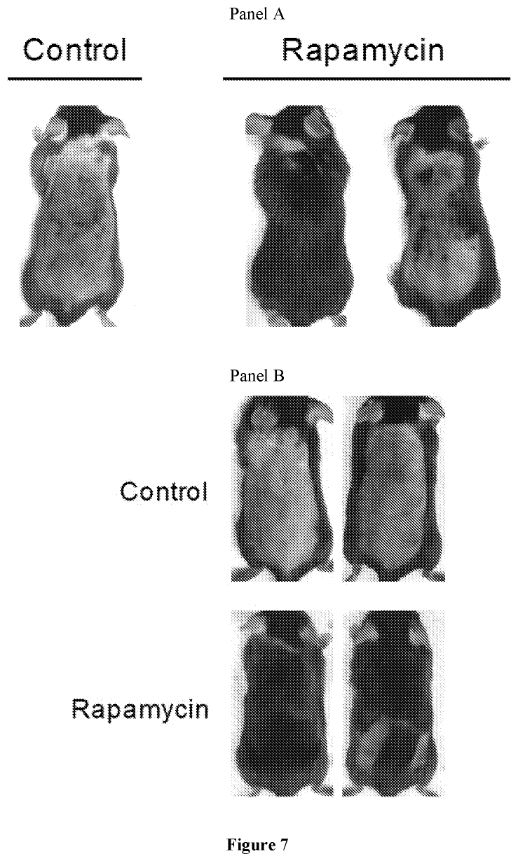

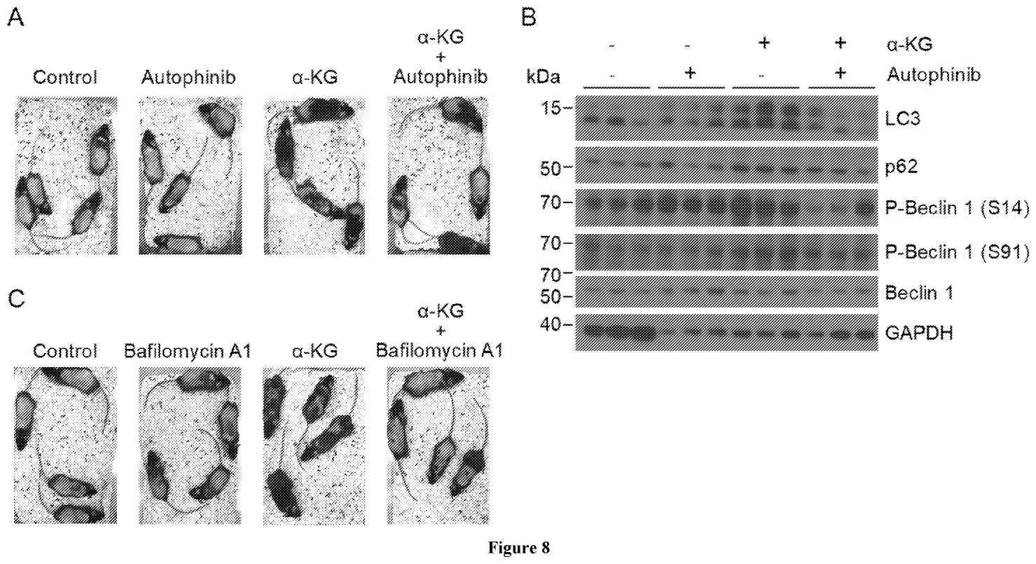

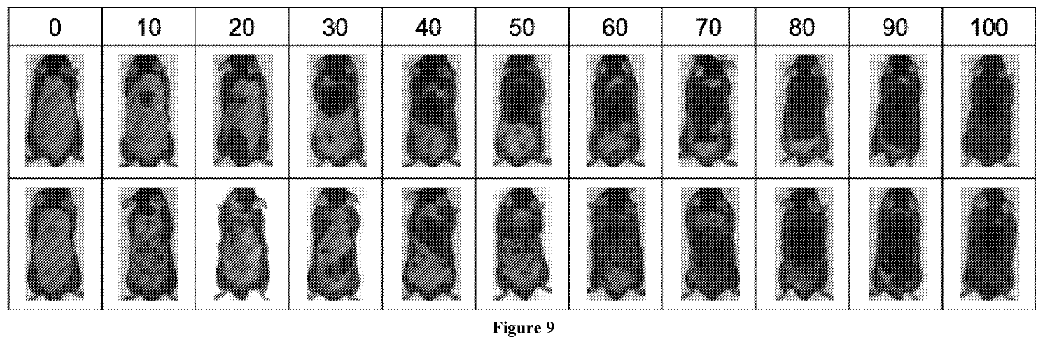

AMP-activated protein kinase (AMPK) is another common downstream effector of α-KG and oligomycin. AMPK, a key cellular energy sensor, is activated by decreases in cellular energy charge, e.g., upon glucose starvation and many other cellular stress conditions. AMPK also elevates autophagy. Consistent with this understanding, AMPK-dependent Beclin 1 phosphorylation on S91 was increased in mouse skin treated with α-KG and oligomycin (FIGS. 1, Panel E and FIG. 2, Panel E). Further, as shown in FIG. 3, Panels A-D, anagen induction and hair regeneration were also stimulated by topical treatment with the AMPK activator, 5-aminoimidazole-4-carboxamide ribonucleotide (AICAR), an AMP analog. Metformin, another agonist of AMPK, similarly induced autophagy and hair regeneration (FIG. 3, Panels E-H).

Mechanistically, metformin has been shown to inhibit mitochondrial complex I in the electron transport chain. Interestingly, long-lived C. elegans mitochondrial mutants accumulate various alpha-keto acid metabolites in the exometabolome. α-ketobutyrate (α-KB) supplementation in drinking water over 30 weeks greatly improved hair coating in old mice (FIG. 3, Panels I-J). In pilot experiments testing topical treatments on aged mice, topical α-KB treatment only moderately promoted hair growth in shaved aged animals, whereas topical α-KG or rapamycin did not visibly increase (or even slightly decreased, if it changed at all) hair regeneration (data not shown). In contrast, in young mice, like α-KG and rapamycin treatments, topical α-KB treatment substantially induced skin pigmentation and hair regeneration (FIG. 3, Panels K-M). Autophagy was also induced, as indicated by elevated LC3 and phosphorylated Beclin 1 in the treated skin (FIG. 3, Panel N).

mTOR has previously been reported to be required for HF SC activation and anagen entry. However, the results disclosed herein indicate that moderate inhibition of mTOR by rapamycin accompanied by autophagy induction stimulates hair regeneration. Such a dichotomy may also exist for mitochondrial regulation. Mitochondrial respiration is required for HF SC cycle and yet genetic perturbation of mitochondrial function abolishes hair regeneration. The experiments showing that oligomycin, a complex V inhibitor, actually promotes hair regeneration suggests that mild mitochondrial inhibition may prove to be beneficial. Since mitochondrial complex V acts upstream of TOR from C. elegans to Drosophila spp. and humans and autophagy is induced both by mitochondrial complex V inhibition and by TOR inhibition (FIG. 2, Panel E), whether autophagy induction alone may be sufficient to elicit hair regeneration was examined.

As disclosed herein, the TOR-independent autophagy inducing small molecule, SMER28, was used to determine whether autophagy induction alone would elicit hair regeneration. Topical SMER28 administration increased autophagic induction of LC3 and p62 in mouse dorsal skin (FIG. 4, Panels A-B, and FIG. 6, Panel F). The level of Beclin 1 Ser14 phosphorylation, which depends on mTOR, was not increased in SMER28-treated skin (FIG. 4, Panel B), indicating an mTOR-independent autophagy-inducing effect by SMER28. Additionally, SMER28 did not appear to induce autophagy by impinging on AMPK, as Beclin 1 S91 phosphorylation was also not increased in SMER28-treated skin (FIG. 4, Panel B). Strikingly, SMER28 also greatly induced hair regeneration (FIG. 4, Panel C). These findings strongly support the role of autophagy in stimulating hair regeneration.

To examine whether autophagy is necessary for SMER28-stimulated hair regeneration, autophinib, which inhibits VPS34 and autophagosome formation, was employed. Co-treatment with autophinib prevented hair regeneration by SMER28 (FIG. 4, Panel D), indicating autophagy is important for hair regeneration. Likewise, autophagy is important for stimulating hair regeneration by α-KG (FIG. 8) as shown by co-treatments with autophinib, as well as with bafilomycin A1, which disrupts autophagic flux by inhibiting vacuolar H(+)-ATPase (V-ATPase)-dependent acidification and Ca-P60A/SERCA-dependent autophagosome-lysosome fusion.

To understand whether autophagy may be integral to the natural hair follicle cycle, autophagy over different hair follicle stages was examined, and autophagy is elevated as hair follicle progresses naturally through anagen; autophagy decreases in catagen and remains low in telogen (FIG. 5).

In summary, the experiments herein show that hair regeneration can be stimulated by inducing autophagy.

Therefore, in some embodiments, the present invention is directed to stimulating hair regeneration in a subject in need thereof, which comprises inducing autophagy in the subject by administering one or more autophagy inducing agents to the subject. As used herein, subjects “in need” includes those who have hair loss as a result of lengthened telogen phase, shortened anagen phase, and/or hampered anagen induction and those who desire hair regeneration, hair growth, and/or improved hair pigmentation. In some embodiments, the present invention is directed to methods for treating, inhibiting, or reducing hair loss; improving or stimulating hair growth; treating, inhibiting, or reducing pigmentation loss; and/or improving or stimulating pigmentation production in a subject which comprises administering the subject one or more autophagy inducing agents. In some embodiments, the present invention is directed to compositions for treating, inhibiting, or reducing hair loss; improving or stimulating hair growth; treating, inhibiting, or reducing pigmentation loss; and/or improving or stimulating pigmentation production in a subject, said compositions comprise a one or more autophagy inducing agents.

In some embodiments, the subject is an animal. In some embodiments, the subject is an animal such as a rodent or a non-human primate. In some embodiments, the subject is a human. In some embodiments, the subject is aging. In some embodiments, the subject is an aged subject. As used herein, a subject who is “aging” refers to a subject in the period of life when untreated control subjects begin to physically, mentally, and/or biologically deteriorate. In some embodiments, a subject who is aging is one whose chronological age is at least at the median point of the average lifespan of untreated control subjects. As used herein, an “aged” subject is one whose chronological age is at least two-thirds the average life expectancy of untreated control subjects. For example, if the average life expectancy of a given strain of a laboratory mouse is 2 years, an aged mouse of that strain is at least 16 months, and if the average life expectancy of another strain of laboratory mouse is 3 years, an aged mouse of that strain is 24 months. For humans, if the average life expectancy of a human is about 80 years, an aged human is about 53 years. It should be noted that a subject who is aging may or may not be an aged subject.

As used herein, an “autophagy inducing agent” refers to compound that induces autophagy as compared to a negative control. Autophagy inducing agents include ATP synthase inhibitors, TOR inhibitors, AMPK activators, and TOR-independent autophagy enhancers. However, explicitly excluded from “autophagy inducing agents”, as defined herein, are alpha-ketobutyrate compounds and glutarate compounds as described in WO2018064468. In some embodiments, two or more autophagy inducing agents are co-administered to the subject. As used herein, “co-administered” refers to the administration of at least two different agents, e.g., a first autophagy inducing agent and a second autophagy inducing agent, to a subject. In some embodiments, the co-administration is concurrent. In embodiments involving concurrent co-administration, the agents may be administered as a single composition, e.g., an admixture, or as two separate compositions. In some embodiments, the first agent is administered before and/or after the administration of the second agent. Where the co-administration is sequential, the administration of the first and second agents may be separated by a period of time, e.g., minutes, hours, or days. Those of skill in the art understand that the formulations and/or routes of administration of the various agents or therapies used may vary. The appropriate dosage for co-administration can be readily determined by one skilled in the art. In some embodiments, when two or more agents are co-administered, the respective agents are administered at lower dosages than appropriate for their administration alone.

As used herein, “ATP synthase inhibitors” refers to compounds that inhibit ATP synthase as compared to a control. Examples of ATP synthase inhibitors include a-helical basic peptide inhibitors, angiostatin, enterostatin, tentoxin, tentoxin analogs, leucinostatins, efrapeptins, stilbenes, flavones, isoflavones, steroidal estradiols, estrogen metabolites, polyketide inhibitors (e.g., macrolides), organotin compounds, α-Pyrone and derivatives thereof, amphiphilic cationic dyes, and the like. See, e.g., Hong, et al., (2008) Microbiology and Molecular Biology Reviews: MIMBR, 72(4): 590-641. In some embodiments, the ATP synthase inhibitor is a macrolide such as an oligomycin (of any type, e.g., A, B, C, D, E, and F), peliomycin, venturicidin (of any type, e.g., A, B, or X), ossamycin, apoptolidin, and cytovaricin. In some embodiments, the ATP synthase inhibitor is an oligomycin. In some embodiments, the oligomycin is oligomycin A.

As used herein, “TOR inhibitors” refers to compounds that inhibit TOR (target of rapamycin) as compared to a control. Examples of TOR inhibitors include rapamycin, rapamycin derivatives (e.g., sirolimus, temsirolimus, everolimus, etc.), dactolisib, GSK2126458, XL765, AZD8055, INK128/MLN0128. OSI027, RapaLinks, and the like. See, e.g., Xie, et al. (2016) F1000Research, 5, F1000 Faculty Rev-2078. In some embodiments, the TOR inhibitor is rapamycin or a rapamycin derivative.

As used herein, “AMPK activators” refers to compounds that activate AMPK (AMP-activated protein kinase) as compared to a control. Examples of AMPK activators include indirect AMPK activators (e.g., the compounds disclosed in WO2009124636, WO2009100130, WO2011029855, WO2011138307, WO2011080277, WO2011032320, WO2011033099), biguanides (e.g., metformin), thiazolidinediones, polyphenols, ginsenosides, α-lipoic acid, direct AMPK activators (e.g., 5-aminoimidazole-4-carboxamide ribonucleotide (AICAR), thienopyridone, benzimidazole, salicylate, Compound-13, PT-1, MT 63-78 (Debio0930)), thienopyridone and derivatives thereof, benzimidazole and derivatives thereof, 5-(5-hydroxyl-isoxazol-3-yl)-furan-2-phosphonic acid (C-2), and the like. See, e.g., Kim, et al. (2016) Experimental & molecular medicine, 48(4): e224. In some embodiments, the AMPK activator is a direct AMPK activator. In some embodiments, the AMPK activator is 5-aminoimidazole-4-carboxamide ribonucleotide (AICAR). In some embodiments, the AMPK activator is a biguanide. In some embodiments, the AMPK activator metformin.

As used herein, “TOR-independent autophagy enhancers” refers to compounds that, independent of the TOR signaling pathway, induce or enhance autophagy as compared to a control. Examples of TOR-independent autophagy enhancers include, SMER28, also included are chloroquine and 3-MA, which induce autophagy in vivo, and the like.

### Compositions

Compositions, including pharmaceutical compositions, comprising one or more autophagy inducing agents are contemplated herein. The term “pharmaceutical composition” refers to a composition suitable for pharmaceutical use in a subject. A pharmaceutical composition generally comprises an effective amount of an active agent, e.g., one or more autophagy inducing agents, and a pharmaceutically acceptable carrier. The term “effective amount” refers to a dosage or amount sufficient to produce a desired result. The desired result may comprise an objective or subjective improvement in the recipient of the dosage or amount, e.g., long-term survival, effective prevention of a disease state, and the like. In addition to the one or more autophagy inducing agents, pharmaceutical compositions may include one or more supplementary agents. Examples of suitable supplementary agents include alpha-ketobutyrate compounds and glutarate compounds as described in WO2018064468, growth factors (e.g., TGF-β2, IGF-1, KGF, HGF), and the like.

In some embodiments, the compositions comprise, consists essentially of, or consist of one or more autophagy inducing agents. As used herein, the phrase “consists essentially of” in the context of a composition consisting essentially of one or more autophagy inducing agents, means that other ingredients that exhibit a biological activity or function other than autophagy may be included so long as the ingredients do not significantly change the activity of the one or more autophagy inducing agents. As used herein, the phrase “consists of” in the context of a composition consisting of one or more autophagy inducing agents means that the composition excludes other ingredients that exhibit a biological activity or function that is intended to have an effect on the subject being treated, e.g., other active pharmaceutical ingredients are excluded, however, the composition may include antibacterial and antifungal agents intended to prevent bacterial and fungal growth in composition itself, carriers, diluents, binders, etc.

The one or more autophagy inducing agents may be administered, preferably in the form of pharmaceutical compositions, to a subject. Preferably the subject is mammalian, more preferably, the subject is human. Preferred pharmaceutical compositions are those comprising at least one autophagy inducing agent in a therapeutically effective amount and a pharmaceutically acceptable vehicle.

As used herein, a “therapeutically effective amount” refers to an amount that may be used to treat, prevent, or inhibit a given disease or condition, such as hair loss, in a subject as compared to a control, such as a placebo. Again, the skilled artisan will appreciate that certain factors may influence the amount required to effectively treat a subject, including the degree of hair loss, previous treatments, the general health and age of the subject, and the like. Nevertheless, therapeutically effective amounts may be readily determined by methods in the art. In some embodiments, a therapeutically effective amount of a autophagy inducing agent ranges from about 0.01 to about 10 mg/kg body weight, about 0.01 to about 3 mg/kg body weight, about 0.01 to about 2 mg/kg, about 0.01 to about 1 mg/kg, or about 0.01 to about 0.5 mg/kg body weight for parenteral formulations. Therapeutically effective amounts for oral administration may be up to about 10-fold higher. It should be noted that treatment of a subject with a therapeutically effective amount may be administered as a single dose or as a series of several doses. The dosages used for treatment may increase or decrease over the course of a given treatment. Optimal dosages for a given set of conditions may be ascertained by those skilled in the art using dosage-determination tests and/or diagnostic assays in the art. Dosage-determination tests and/or diagnostic assays may be used to monitor and adjust dosages during the course of treatment.

Pharmaceutical compositions may be formulated for the intended route of delivery, including intravenous, intramuscular, intra peritoneal, subcutaneous, intraocular, intrathecal, intraarticular, intrasynovial, cisternal, intrahepatic, intralesional injection, intracranial injection, infusion, and/or inhaled routes of administration using methods known in the art. Pharmaceutical compositions may include one or more of the following: pH buffered solutions, adjuvants (e.g., preservatives, wetting agents, emulsifying agents, and dispersing agents), liposomal formulations, nanoparticles, dispersions, suspensions, or emulsions, as well as sterile powders for reconstitution into sterile injectable solutions or dispersions. The compositions and formulations may be optimized for increased stability and efficacy using methods in the art. See, e.g., Carra et al., (2007) Vaccine 25:4149-4158.

The compositions may be administered to a subject by any suitable route including oral, transdermal, subcutaneous, intranasal, inhalation, intramuscular, and intravascular administration. It will be appreciated that the preferred route of administration and pharmaceutical formulation will vary with the condition and age of the subject, the nature of the condition to be treated, the therapeutic effect desired, and the particular autophagy inducing agent used. In some embodiments, the one or more autophagy inducing agents are topically administered to the site to be treated on the subject.

As used herein, a “pharmaceutically acceptable vehicle” or “pharmaceutically acceptable carrier” are used interchangeably and refer to solvents, dispersion media, coatings, antibacterial and antifungal agents, isotonic and absorption delaying agents, and the like, that are compatible with pharmaceutical administration and comply with the applicable standards and regulations, e.g., the pharmacopeial standards set forth in the United States Pharmacopeia and the National Formulary (USP-NF) book, for pharmaceutical administration. Thus, for example, unsterile water is excluded as a pharmaceutically acceptable carrier for, at least, intravenous administration. Pharmaceutically acceptable vehicles include those known in the art. See, e.g., Remington: The Science and Practice of Pharmacy 20th ed (2000) Lippincott Williams & Wilkins, Baltimore, Md.

The pharmaceutical compositions may be provided in dosage unit forms. As used herein, a “dosage unit form” refers to physically discrete units suited as unitary dosages for the subject to be treated; each unit containing a predetermined quantity of the one or more autophagy inducing agent calculated to produce the desired therapeutic effect in association with the required pharmaceutically acceptable carrier. The specification for the dosage unit forms of the invention are dictated by and directly dependent on the unique characteristics of the given autophagy inducing agent and desired therapeutic effect to be achieved, and the limitations inherent in the art of compounding such an active compound for the treatment of individuals.

Toxicity and therapeutic efficacy of autophagy inducing agents according to the instant invention and compositions thereof can be determined using cell cultures and/or experimental animals and pharmaceutical procedures in the art. For example, one may determine the lethal dose, LC₅₀ (the dose expressed as concentration×exposure time that is lethal to 50% of the population) or the LD₅₀ (the dose lethal to 50% of the population), and the ED₅₀ (the dose therapeutically effective in 50% of the population) by methods in the art. The dose ratio between toxic and therapeutic effects is the therapeutic index and it can be expressed as the ratio LD₅₀ /ED₅₀. autophagy inducing agents which exhibit large therapeutic indices are preferred. While autophagy inducing agents that result in toxic side-effects may be used, care should be taken to design a delivery system that targets such compounds to the site of treatment to minimize potential damage to uninfected cells and, thereby, reduce side-effects.

The data obtained from the cell culture assays and animal studies can be used in formulating a range of dosages for use in humans. Preferred dosages provide a range of circulating concentrations that include the ED₅₀ with little or no toxicity. The dosage may vary depending upon the dosage form employed and the route of administration utilized. Therapeutically effective amounts and dosages of one or more autophagy inducing agents can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography. Additionally, a dosage suitable for a given subject can be determined by an attending physician or qualified medical practitioner, based on various clinical factors.

### Kits

In some embodiments, the present invention provides kits comprising one or more autophagy inducing agents, optionally in a composition or in combination with one or more supplementary agents, packaged together with one or more reagents or drug delivery devices for preventing, inhibiting, reducing, or treating hair loss in a subject. In some embodiments, the kits comprise the one or more autophagy inducing agents, optionally in one or more unit dosage forms, packaged together as a pack and/or in drug delivery device, e.g., a pre-filled syringe.

In some embodiments, the kits include a carrier, package, or container that may be compartmentalized to receive one or more containers, such as vials, tubes, and the like. In some embodiments, the kits optionally include an identifying description or label or instructions relating to its use. In some embodiments, the kits include information prescribed by a governmental agency that regulates the manufacture, use, or sale of compounds and compositions as contemplated herein.

The following examples are intended to illustrate but not to limit the invention.

### EXAMPLES—METHODS

### Assay for Hair Regeneration in Mice

All compounds were tested in both male and female mice. Every experiment was repeated independently at least 2 times. Some treatments with different agents were performed concurrently with shared control arms. C57BL/6J male mice were obtained at 6 or 8 weeks of age from Jackson Laboratories (Bar Harbor, ME). C57BL/6J female mice were obtained at 8 weeks of age from Jackson Laboratories (Bar Harbor, Me.). Mice were fed a standard chow diet and provided ad libitum access to food and water throughout the study. Mice were shaved dorsally in telogen, i.e., postnatal days of about 43-45 for males (unless otherwise indicated) and Day 58 for females, respectively. Vehicle control (25 μL DMSO, unless otherwise indicated) or test compounds (in 25 μL DMSO, unless otherwise indicated) were topically applied on the shaved skin every other day (unless otherwise described) for the duration of the experiments (3-6 weeks). Appearance of skin pigmentation and hair growth were monitored and documented by photos and videos. Progression was also assigned a value from 0 to 100 based on pigmentation levels and hair shaft density, with 0 indicating no hair growth (and no pigmentation) and higher number corresponding to darker skin and larger areas of dense hair growth. Images representing different scores are presented in FIG. 9. α-KG (Sigma, 75890), oligomycin (Cell Signaling, 9996L), rapamycin (Selleckchem, S1039), AICAR (Selleckchem, S1802), metformin (Sigma, PHR1084), α-KB (Sigma, K401), SMER28 (Selleckchem, S8240), autophinib (Selleckchem, S8596), bafilomycin A1 (Selleckchem, S1413), or indicated combinations in about 250 μL Premium Lecithin Organogel (PLO) Base (Transderma Pharmaceuticals Inc.) were used for each mouse. The vehicle DMSO was also mixed with PLO base for topical application. The timing of the hair cycle was not altered using PLO base+DMSO vs PLO base alone.

### Aged Mice

For oral α-KB treatment, aged male and female C57BL/6J mice were obtained at 87 weeks of age (NIA aged rodent colonies). Mice were housed in a controlled SPF facility (22±2° C., 6:00-18:00, 12 h/12 h light/dark cycle) at UCLA. Mice were fed a standard chow diet and provided ad libitum access to food and water throughout the study. Treatment with either water (vehicle control), or α-KB (90 mg/kg bodyweight) in drinking water, started when mice were at 101 weeks of age. For topical α-KB treatment, aged male C57BL/6J mice were obtained at 21 months of age (NIA aged rodent colonies), shaved the following week, and topically treated with α-KB (32 mM) every other day for one month. All experiments were approved by the UCLA Chancellor's Animal Research Committee.

### Histology and Microscopy

Mouse dorsal skin was shaved before being collected for histological and molecular analyses. Full-thickness skin tissue was then fixed in 10% formalin solution (Sigma, HT501128) overnight and dehydrated for embedding in paraffin. 5 μm paraffin sections were subjected to hematoxylin/eosin staining and immunohistochemistry for Ki-67 (Cell Signaling, 12202), IL-6 (Abcam, ab6672) or F4/80 (Bio-Rad, MCA497G). Images were captured by Leica Aperio ScanScope AT brightfield system at ×20 magnification.

### Western Blotting

Male mice were shaved and treated every other day starting on Postnatal Day 43. After 5 days, telogen skin samples were harvested and stage confirmed. Mouse skin tissue lysate was prepared by homogenization in T-PER Tissue Protein Extraction Buffer (Thermo Scientific, 78510) with protease inhibitors (Roche, 11836153001) and phosphatase inhibitors (Sigma, P5726) by FastPrep-24 (MP Biomedicals). Tissue and cell debris were removed by centrifugation and the lysate was boiled for 5 min in 1×SDS loading buffer containing 5% β-mercaptoethanol. Samples were then subjected to SDS-PAGE on NuPAGE Novex 12% Bis-Tris gels (Invitrogen, NP0343BOX), and western blotting was carried out with antibodies against LC3 (Novus, NB100-2220), p62 (Sigma, P0068), Phospho-Beclin-1 (Ser15) (corresponding to Ser14 in mouse) (Cell Signaling, 84966), Phospho-Beclin-1 (Ser93) (corresponding to Ser91 in mouse) (Cell Signaling, 14717), Beclin-1 (Abcam, ab207612), or GAPDH (Ambion, AM4300).

### Quantitative Reverse Transcription PCR (RT-qPCR)

At 24 hours post-treatment, telogen skin samples were harvested and total RNA was isolated using TRIzol reagent (Invitrogen) from whole thickness mouse skin tissue. cDNA was synthesized using iScript Reverse Transcription Supermix (Bio-Rad). iTaq Universal SYBR Green Supermix (Bio-Rad) and a Bio-Rad CFX Connect instrument were used for quantitative RT-PCR. The primer sequences used for RT-qPCR are as follows:

p62 forward:
SEQ ID NO: 1:
GAAGAATGTGGGGGAGAGTGTGG
p62 reverse:
SEQ ID NO: 2:
TGCCTGTGCTGGAACTTTCTGG
B2m forward:
SEQ ID NO: 3:
CAGCATGGCTCGCTCGGTGAC
B2m reverse:
SEQ ID NO: 4:
CGTAGCAGTTCAGTATGTTCG

### Statistical Analysis

All treatments were repeated at least two times. Data represent biological replicates. Appropriate statistical tests were used for every Figure. Data meet the assumptions of the statistical tests described for each Figure. Mean±s.d. is plotted in all Figures.

### REFERENCES

To the extent necessary, the following references are herein incorporated by reference:

1. Barzilai, N., et al. (2016). “Metformin as a Tool to Target Aging.” Cell Metab 23(6): 1060-1065.

2. Belleudi, F., et al. (2014). “FGF7/KGF regulates autophagy in keratinocytes: A novel dual role in the induction of both assembly and turnover of autophagosomes.” Autophagy 10(5): 803-821.

3. Burkewitz, K., et al. (2014). “AMPK at the nexus of energetics and aging.” Cell Metab 20(1): 10-25.

4. Butler, J. A., et al. (2010). “Long-lived mitochondrial (Mit) mutants of Caenorhabditis elegans utilize a novel metabolism.” FASEB J 24(12): 4977-4988.

5. Castilho, R. M., et al. (2009). “mTOR mediates Wnt-induced epidermal stem cell exhaustion and aging.” Cell Stem Cell 5(3): 279-289.

6. Chikh, A., et al. (2014). “iASPP is a novel autophagy inhibitor in keratinocytes.” J Cell Sci 127(Pt 14): 3079-3093.

7. Chin, R. M., et al. (2014). “The metabolite alpha-ketoglutarate extends lifespan by inhibiting ATP synthase and TOR.” Nature 509(7505): 397-401.

8. Chueh, S. C., et al. (2013). “Therapeutic strategy for hair regeneration: hair cycle activation, niche environment modulation, wound-induced follicle neogenesis, and stem cell engineering.” Expert Opin Biol Ther 13(3): 377-391.

9. Cuervo, A. M. and J. F. Dice (2000). “Age-related decline in chaperone-mediated autophagy.” J Biol Chem 275(40): 31505-31513.

10. Deng, Z., et al. (2015). “mTOR signaling promotes stem cell activation via counterbalancing BMP-mediated suppression during hair regeneration.” J Mol Cell Biol 7(1): 62-72.

11. Fernandez, A. F., et al. (2018). “Disruption of the beclin 1-BCL2 autophagy regulatory complex promotes longevity in mice.” Nature 558(7708): 136-140.

12. Forni, M. F., et al. (2017). “Caloric Restriction Promotes Structural and Metabolic Changes in the Skin.” Cell Rep 20(11): 2678-2692.

13. Fu, X., et al. (2015). “2-Hydroxyglutarate Inhibits ATP Synthase and mTOR Signaling.” Cell Metab 22(3): 508-515.

14. Galluzzi, L., et al. (2014). “Metabolic control of autophagy.” Cell 159(6): 1263-1276.

15. Gilhar, A., et al. (2012). “Alopecia areata.” N Engl J Med 366(16): 1515-1525.

16. Hamanaka, R. B., et al. (2013). “Mitochondrial reactive oxygen species promote epidermal differentiation and hair follicle development.” Sci Signal 6(261): ra8.

17. Hansen, M., et al. (2008). “A role for autophagy in the extension of lifespan by dietary restriction in C. elegans.” PLoS Genet 4(2): e24.

18. Hardie, D. G., et al. (2012). “AMPK: a nutrient and energy sensor that maintains energy homeostasis.” Nat Rev Mol Cell Biol 13(4): 251-262.

19. Harel, S., et al. (2015). “Pharmacologic inhibition of JAK-STAT signaling promotes hair growth.” Sci Adv 1(9): e1500973.

20. Hirano, T., et al. (1990). “Biological and clinical aspects of interleukin 6.” Immunol Today 11(12): 443-449.

21. Ho, T. T., et al. (2017). “Autophagy maintains the metabolism and function of young and old stem cells.” Nature 543(7644): 205-210.

22. Huang, J., et al. (2016). Ketobutyrate compounds and compositions for treating age-related symptoms and diseases. Google Patents, Google Patents.

23. Ito, M., et al. (2007). “Wnt-dependent de novo hair follicle regeneration in adult mouse skin after wounding.” Nature 447(7142): 316-320.

24. Jia, K. and B. Levine (2007). “Autophagy is required for dietary restriction-mediated life span extension in C. elegans.” Autophagy 3(6): 597-599.

25. Kabeya, Y., et al. (2000). “LC3, a mammalian homologue of yeast Apg8p, is localized in autophagosome membranes after processing.” EMBO J 19(21): 5720-5728.

26. Kellenberger, A. J. and M. Tauchi (2013). “Mammalian target of rapamycin complex 1 (mTORC1) may modulate the timing of anagen entry in mouse hair follicles.” Exp Dermatol 22(1): 77-80.

27. Keyes, B. E., et al. (2013). “Nfatc1 orchestrates aging in hair follicle stem cells.” Proc Natl Acad Sci USA 110(51): E4950-4959.

28. Kim, J., et al. (2013). “Differential regulation of distinct Vps34 complexes by AMPK in nutrient stress and autophagy.” Cell 152(1-2): 290-303.

29. Kloepper, J. E., et al. (2015). “Mitochondrial function in murine skin epithelium is crucial for hair follicle morphogenesis and epithelial-mesenchymal interactions.” J Invest Dermatol 135(3): 679-689.

30. Knowler, W. C., et al. (2002). “Reduction in the incidence of type 2 diabetes with lifestyle intervention or metformin.” N Engl J Med 346(6): 393-403.

31. Levine, B. and G. Kroemer (2008). “Autophagy in the pathogenesis of disease.” Cell 132(1): 27-42.

32. Liang, X. H., et al. (1999). “Induction of autophagy and inhibition of tumorigenesis by beclin 1.” Nature 402(6762): 672-676.

33. Magerl, M., et al. (2001). “Patterns of proliferation and apoptosis during murine hair follicle morphogenesis.” J Invest Dermatol 116(6): 947-955.

34. Mauvezin, C. and T. P. Neufeld (2015). “Bafilomycin Al disrupts autophagic flux by inhibiting both V-ATPase-dependent acidification and Ca-P60A/SERCA-dependent autophagosome-lysosome fusion.” Autophagy 11(8): 1437-1438.

35. Messenger, A. G. and J. Rundegren (2004). “Minoxidil: mechanisms of action on hair growth.” Br J Dermatol 150(2): 186-194.

36. Mizushima, N., et al. (2008). “Autophagy Fights disease through cellular self-digestion.” Nature 451(7182): 1069-1075.

37. Muller-Rover, S., et al. (2001). “A comprehensive guide for the accurate classification of murine hair follicles in distinct hair cycle stages.” J Invest Dermatol 117(1): 3-15.

38. Ohsumi, Y. (2014). “Historical landmarks of autophagy research.” Cell Res 24(1): 9-23.

39. Ou, H. T., et al. (2016). “Metformin improved health-related quality of life in ethnic Chinese women with polycystic ovary syndrome.” Health Qual Life Outcomes 14(1): 119.

40. Owen, M. R., et al. (2000). “Evidence that metformin exerts its anti-diabetic effects through inhibition of complex 1 of the mitochondrial respiratory chain.” Biochem J 348 Pt 3: 607-614.

41. Parodi, C., et al. (2018). “Autophagy is essential for maintaining the growth of a human (mini-)organ: Evidence from scalp hair follicle organ culture.” PLoS Biol 16(3): e2002864.

42. Petukhova, L., et al. (2010). “Genome-wide association study in alopecia areata implicates both innate and adaptive immunity.” Nature 466(7302): 113-117.

43. Robke, L., et al. (2017). “Phenotypic Identification of a Novel Autophagy Inhibitor Chemotype Targeting Lipid Kinase VPS34.” Angew Chem Int Ed Engl 56(28): 8153-8157.

44. Russell, R. C., et al. (2013). “ULK1 induces autophagy by phosphorylating Beclin-1 and activating VPS34 lipid kinase.” Nat Cell Biol 15(7): 741-750.

45. Sahani, M. H., et al. (2014). “Expression of the autophagy substrate SQSTM1/p62 is restored during prolonged starvation depending on transcriptional upregulation and autophagy-derived amino acids.” Autophagy 10(3): 431-441.

46. Salemi, S., et al. (2012). “Autophagy is required for self-renewal and differentiation of adult human stem cells.” Cell Res 22(2): 432-435.

47. Sarkar, S., et al. (2007). “Small molecules enhance autophagy and reduce toxicity in Huntington's disease models.” Nat Chem Biol 3(6): 331-338.

48. Schneider, M. R., et al. (2009). “The hair follicle as a dynamic miniorgan.” Curr Biol 19(3): R132-142.

49. Shahebrahimi, K., et al. (2016). “Comparison clinical and metabolic effects of metformin and pioglitazone in polycystic ovary syndrome.” Indian J Endocrinol Metab 20(6): 805-809.

50. Shyh-Chang, N., et al. (2013). “Lin28 enhances tissue repair by reprogramming cellular metabolism.” Cell 155(4): 778-792.

51. Slominski, A. and R. Paus (1993). “Melanogenesis is coupled to murine anagen: toward new concepts for the role of melanocytes and the regulation of melanogenesis in hair growth.” J Invest Dermatol 101(1 Suppl): 90S-97S.

52. Solanas, G., et al. (2017). “Aged Stem Cells Reprogram Their Daily Rhythmic Functions to Adapt to Stress.” Cell 170(4): 678-692 e620.

53. Sun, X., et al. (2014). “A mitochondrial ATP synthase subunit interacts with TOR signaling to modulate protein homeostasis and lifespan in Drosophila.” Cell Rep 8(6): 1781-1792.

54. Tang, A. H. and T. A. Rando (2014). “Induction of autophagy supports the bioenergetic demands of quiescent muscle stem cell activation.” Embo Journal 33(23): 2782-2797.

55. Walpoth, B. H., et al. (2001). “Prevention of neointimal proliferation by immunosuppression in synthetic vascular grafts.” Eur J Cardiothorac Surg 19(4): 487-492.

56. Wang, H., et al. (2006). “Activated macrophages are essential in a murine model for T cell-mediated chronic psoriasiform skin inflammation.” J Clin Invest 116(8): 2105-2114.

57. Wang, L., et al. (2015). “Oxidative stress and substance P mediate psychological stress-induced autophagy and delay of hair growth in mice.” Arch Dermatol Res 307(2): 171-181.

58. Wheaton, W. W., et al. (2014). “Metformin inhibits mitochondrial complex I of cancer cells to reduce tumorigenesis.” Elife 3: e02242.

59. Yoshihara, N., et al. (2015). “The significant role of autophagy in the granular layer in normal skin differentiation and hair growth.” Arch Dermatol Res 307(2): 159-169.

60. Zhang, C. S., et al. (2017). “Fructose-1,6-bisphosphate and aldolase mediate glucose sensing by AMPK.” Nature 548(7665): 112-116.

All scientific and technical terms used in this application have meanings commonly used in the art unless otherwise specified.

As used herein, the terms “subject”, “patient”, and “individual” are used interchangeably to refer to humans and non-human animals. The term “non-human animal” includes all vertebrates, e.g., mammals and non-mammals, such as non-human primates, horses, sheep, dogs, cows, pigs, chickens, and other veterinary subjects and test animals. In some embodiments, the subject is a mammal. In some embodiments, the subject is a human.

As used herein, the term “diagnosing” refers to the physical and active step of informing, i.e., communicating verbally or by writing (on, e.g., paper or electronic media), another party, e.g., a patient, of the diagnosis. Similarly, “providing a prognosis” refers to the physical and active step of informing, i.e., communicating verbally or by writing (on, e.g., paper or electronic media), another party, e.g., a patient, of the prognosis.

The use of the singular can include the plural unless specifically stated otherwise. As used in the specification and the appended claims, the singular forms “a”, “an”, and “the” can include plural referents unless the context clearly dictates otherwise.

As used herein, “and/or” means “and” or “or”. For example, “A and/or B” means “A, B, or both A and B” and “A, B, C, and/or D” means “A, B, C, D, or a combination thereof” and said “A, B, C, D, or a combination thereof” means any subset of A, B, C, and D, for example, a single member subset (e.g., A or B or C or D), a two-member subset (e.g., A and B; A and C; etc.), or a three-member subset (e.g., A, B, and C; or A, B, and D; etc.), or all four members (e.g., A, B, C, and D).

As used herein, the phrase “one or more of”, e.g., “one or more of A, B, and/or C” means “one or more of A”, “one or more of B”, “one or more of C”, “one or more of A and one or more of B”, “one or more of B and one or more of C”, “one or more of A and one or more of C” and “one or more of A, one or more of B, and one or more of C”.

The phrase “comprises, consists essentially of, or consists of A” is used as a tool to avoid excess page and translation fees and means that in some embodiments the given thing at issue: comprises A, consists essentially of A, or consists of A. For example, the sentence “In some embodiments, the composition comprises, consists essentially of, or consists of A” is to be interpreted as if written as the following three separate sentences: “In some embodiments, the composition comprises A. In some embodiments, the composition consists essentially of A. In some embodiments, the composition consists of A.”

Similarly, a sentence reciting a string of alternates is to be interpreted as if a string of sentences were provided such that each given alternate was provided in a sentence by itself. For example, the sentence “In some embodiments, the composition comprises A, B, or C” is to be interpreted as if written as the following three separate sentences: “In some embodiments, the composition comprises A. In some embodiments, the composition comprises B. In some embodiments, the composition comprises C.” As another example, the sentence “In some embodiments, the composition comprises at least A, B, or C” is to be interpreted as if written as the following three separate sentences: “In some embodiments, the composition comprises at least A. In some embodiments, the composition comprises at least B. In some embodiments, the composition comprises at least C.”

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

1. A method for improving or stimulating hair regeneration; treating, inhibiting, or reducing hair loss; improving or stimulating hair growth; treating, inhibiting, or reducing pigmentation loss; and/or improving or stimulating pigmentation production in a subject which comprises administering to the subject one or more autophagy inducing agents.

2. The method according to claim 1, wherein the one or more autophagy inducing agents are an ATP synthase inhibitor, a TOR inhibitor, an AMPK activator, and/or a TOR-independent autophagy enhancer.

3. The method according to claim 1, wherein the one or more autophagy inducing agents are a macrolide such as an oligomycin.

4. The method according to claim 1, wherein the one or more autophagy inducing agents are rapamycin or a rapamycin derivative.

5. The method according to claim 1, wherein the one or more autophagy inducing agents are a biguanide such as metformin.

6. The method according to claim 1, wherein the one or more autophagy inducing agents are 5-aminoimidazole-4-carboxamide ribonucleotide (AICAR).

7. The method according to claim 1, wherein the one or more autophagy inducing agents are SMER28.

8. The method according to claim 1, wherein the one or more autophagy inducing agents are selected from the group consisting of: a macrolide such as an oligomycin, rapamycin or a rapamycin derivative, a biguanide such as metformin, 5-aminoimidazole-4-carboxamide ribonucleotide (AICAR), SMER28, and any combination thereof.

9. The method of claim 1, wherein the one or more autophagy inducing agents are formulated for oral, parenteral, or topical administration.

10. The method of claim 1, wherein the one or more autophagy inducing agents are formulated as a gel, a cream, an ointment, a paste, or a lotion.
